(19) United States
(12) Patent Application Publication
(10) Pub. No.: US 2011/0028835 A1
(43) Pub. Date: Feb. 3, 2011

(54) ULTRASONIC WAVE IRRADIATION DEVICE

(76) Inventor: Kenichi Kawabata, Kodaira (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/936,260

(22) PCT Filed: Feb. 20, 2009
(86) PCT No.: PCT/JP2009/000723

§ 371 Date: Oct. 4, 2010

(30) Foreign Application Priority Data
Apr. 4, 2008 (JP) 2008-097704

(51) Int. Cl. A61B 8/00

(52) U.S. Cl. 600/431

(57) ABSTRACT

Disclosed is an ultrasonic wave irradiation device usable for the application of a phase change type ultrasonic wave contrast medium, in which any interference between ultrasonic wave irradiation for the phase change application and ultrasonic wave irradiation for treatment application does not occur. An ultrasonic wave with a high frequency and a large magnitude of the maximum negative pressure is used for the phase change application, and an ultrasonic wave with a lower frequency and a large magnitude of the maximum positive pressure is used for the treatment application.

### TECHNICAL FIELD

The present invention relates to an ultrasonic wave irradiation method and irradiation device for diagnosis or treatment employing a phase change type ultrasonic wave contrast medium.

### BACKGROUND ART

Many years have passed since diagnostic imaging modalities including X-ray CT, MRI, and ultrasonic diagnosis apparatuses become mandatory tools in medical practice. The modalities image a difference in a CT number, a spin relaxation time, or an acoustic impedance inside a living body. Since the difference in a physical property reflects the structure (shape) of the living body, the modalities are called “anatomical imaging.” In contrast, a modality of imaging certain regions that structurally belong to the same tissue but are in functionally different states is called “functional imaging.” In the functional imaging, a modality of visualizing molecular biological information, that is, an existing state of molecules constituting a living body, such as, protein, amino acid, or nucleic acid is often called “molecular imaging.” The molecular imaging is expected to be applied to explanation of vital phenomena such as generation and differentiation, or to diagnosis or treatment of diseases, and is therefore one of research horizons currently most greatly attracting attention. In the molecular imaging, a “molecular probe” that is a material structured to exhibit selectivity to the molecules constituting a living body is often employed. In this case, a structure detectable by a physical means is appended to the molecular probe in order to visualize an intracorporeal distribution of molecular probes. For example, non-patent document 1 describes an example of the molecular probe to be employed when a tumor is a target. Peptide and an antibody are major molecular probes.

A positron emission topography (PET) apparatus and an optical imaging apparatus are cited as imaging apparatuses specialized in the molecular imaging. The former one is widely adopted as a tool for classifying a degree of spread of a tumor or a degree of progress (stage) thereof in clinical practice, and the latter is widely adopted as a non-invasive pharmacokinetic analysis tool using a small animal for development of a drug or the like. Aside from the apparatuses specialized in the molecular imaging, development of systems, which detect or diagnose diseases in an earlier stage than conventionally, on the basis of an existing modality such as MRI or ultrasound employed in anatomical imaging has been in progress. Out of the systems, the system employing ultrasound has features which the other modalities do not have, such as, (1) excellence in a real-time property, (2) a few restrictions on use in an operation room because of a compact design, and (3) usability not only as a diagnostic tool but also as a treatment tool, and is therefore expected to be an integrated diagnosis/treatment tool usable in any hospital other than large-scale hospitals.

Patent document 1: WO 98/01131

Patent document 2: JP-A-18-320405

Non-patent document 1: Nature Rev. Cancer 2 (750-763, 2002) by Allen

Non-patent document 2: Ultrasound Med. Biol. 27 (1399-1412, 2001) by Holt et al.

Non-patent document 3: J. Acoust. Soc. Am. 88 (2059-2069, 1990) by Holland et al.

Non-patent document 4: Proc. 4th Intern. Symp. Ultrasound Contrast Img. 92 (2004) by Kawabata et al.

Non-patent document 5: IEEE Trans. Medical Imag. 23 (1087-1093, 2004) by Alizad et al.

### DISCLOSURE OF THE INVENTION

### Problems to be Solved by the Invention

In principle, ultrasound as a treatment tool enables least invasive treatment owing to region selectivity due to irradiation of convergent ultrasonic waves from a region separated far from a lesion. In particular, what has attracted attention in recent years is heat coagulation treatment of causing coagulation necrosis to a tissue by raising the temperature of an object region to protein denaturation temperature (approximately 65° C.) or more for a short period of time of 1 min or less. Since the treatment employs high-intensity convergent ultrasonic waves (or high-intensity focused ultrasound (HIFU)) of 1 kW/cm² or more, the treatment is called HIFU treatment. In the HIFU treatment, treatment region selectivity is obtained with the convergence of ultrasonic waves alone. Therefore, if a sight goes wrong because of a body motion or the like, ultrasonic waves of as high intensity as 1 kW/cm² or more are irradiated to a region other than an object of treatment. If an especially significant organ is located in the vicinity of the object of treatment, precautions have to be taken in order to avoid a side effect.

For highly safe treatment, a treatment method having, region selectivity due to, in addition to the convergence of ultrasonic waves, the other mechanism is preferred. For ensuring the selectivity due to anything other than ultrasonic waves, concurrent use of a drug is studied. In particular, a technique using microbubbles, which are often used as an ultrasonic wave contrast medium, as a treatment drug is highly expected. For example, as described in non-patent document 2, a phenomenon that when microbubbles exist in a system, if ultrasonic waves are irradiated, a nominal ultrasonic energy absorption coefficient gets higher has experimentally been verified. If microbubbles can be localized at an object-of-treatment region alone, an intended region alone can be selectively heated by utilizing the phenomenon. When the phenomenon is utilized, such a treatment method is conceivable that a heat coagulation action at an intended region is intensified through ultrasonic irradiation at the intensity for normal heat coagulation on the order of kW/cm², or the heat coagulation action is induced only at the intended region using ultrasonic intensity lower than that for the normal heat coagulation treatment, but a heat coagulation effect is hardly exerted at any other region. As mentioned above, when the existence of microbubbles is utilized in addition to the convergence of ultrasonic waves, compared with when only the convergence of ultrasonic waves is utilized, high region selectivity can be provided in principle. However, the microbubbles can exist only in blood vessels because of a restriction due to the size. It is also hard to allow the microbubbles to exist in distal vessels of small bloodstreams at a high concentration. There is therefore difficulty in localizing the microbubbles at a specific region in a tissue beyond the distal vessel.

As an ultrasonic living-body action involving microbubbles, an action dependent on (acoustic) cavitation is cited. Cavitation is originally a phenomenon that bubble nuclei are produced by ultrasonic waves, and the bubbles grow to end with a collapse. Existence of the microbubbles in a system is equivalent to attainment of a stage, in which the bubbles have grown, with the beginning of the process of cavitation omitted. In the state, when ultrasonic waves are irradiated, a step of nucleation necessary to production of cavitation can be omitted. Since production of bubble nuclei can be omitted, when the microbubbles exist, it is known as described in, for example, non-patent document 3 that acoustic intensity necessary for cavitation production decreases. Irrespective of whether the microbubbles exist from the beginning, once cavitation occurs, high temperature of several thousands of degrees and high pressure of several hundreds of atmospheres are generated at the step of a compression failure of the bubbles. A living-body action is known to occur due directly to the generation or due to an effect of a chemical material called a sonochemical active material like the one described in, for example, patent document 1. In particular, application of cell death or tissue destruction to treatment in a region in which cavitation has caused production is expected.

For example, as described in non-patent document 4, it has been studied to adopt as a contrast medium for ultrasonic diagnosis a drug that takes on nano-sized droplets when being administered to a living body, makes a phase change along with ultrasonic irradiation, and then produces microbubbles. Since the nano-sized droplets are smaller in size than the microbubbles, the droplets can be leaked out from a blood vessel to a tissue such as a tumor or can be highly densely accumulated in a distal blood vessel. When the aforesaid molecular imaging technique of appending a molecular probe is employed, tissue selectivity can be provided. Using the phase-change type contrast medium, ultrasonic contrast enhancement with high tissue selectivity is enabled. Further, as mentioned previously, since the microbubbles produced after a phase change facilitate a heat coagulation action at a region near the microbubbles themselves, once the phase change is induced in a target region alone, a region highly sensitive to the ultrasonic heating action can be obtained. Thus, an integrated region selective diagnosis/treatment system can be, in principle, constructed owing to the phase change and succeeding ultrasonic heat coagulation treatment.

However, for realization of the integrated diagnosis/treatment system employing the phase change contrast medium, it is preferred that: a phase change ultrasonic sequence and a treatment ultrasonic sequence are independent of each other; a treatment effect is not exerted by phase-change ultrasonic waves; and a phase change is not induced by treatment ultrasonic waves. After a phase change is selectively induced at a region that is an object of treatment, when treatment of the region proceeds, if a situation in which the phase change is secondarily induced by the treatment ultrasonic waves takes place, the localizability of the place of existence of the microbubbles derived from the phase change is lost. Accordingly, a region whose sensitivity to the ultrasonic heating action is upgraded by the microbubbles gets wider than an originally anticipated place. The same applies to the reverse situation. Once a treatment effect is exerted by phase-change ultrasonic waves, a region that is originally not an object of treatment is treated because a sight of phase-change ultrasonic waves goes wrong. Since pulses shorter than those for treatment are adopted as the phase-change ultrasonic waves, a possibility that a treatment effect requiring accumulation of energy is exerted by the phase-change ultrasonic waves is low. Therefore, it poses a critical problem that a phase change is secondarily induced by the treatment ultrasonic waves. For example, patent document 2 has disclosed an apparatus that integrates contrast enhancement of an intended region with a phase change contrast medium, and treatment succeeding the contrast enhancement. Even in the apparatus, if a phase change is secondarily induced by treatment ultrasonic waves irradiated after contrast enhancement, selectivity of a region to be treated is degraded.

As mentioned above, a phase change type contrast medium of nano-particles enables tissue selective ultrasonic contrast enhancement. However, when an ordinary irradiation method is adopted, there arises a problem that if a phase change is secondarily induced with succeeding irradiation of treatment ultrasonic waves, selectivity of a region to be treated is not ensured. In particular, when a deep region is an object of treatment, since ultrasonic intensity gets higher at a region near an ultrasonic irradiation source than it does at a focus, the problem is especially severe. An object of the present invention is to provide a safe ultrasonic wave irradiation device that can efficiently perform contrast enhancement with a phase change type ultrasonic wave contrast medium but does not allow a phase change to be secondarily induced by treatment ultrasonic waves.

### Means for Solving the Problems

The present inventor et al. have comparatively studied a property of ultrasonic waves necessary to diagnosis, that is, production of microbubbles due to a phase change of nano-droplets, and a property of the ultrasonic waves necessary to treatment, that is, heat coagulation treatment, and have conceived a technique controlled for each properties not interferes with each other. To begin with, in heat coagulation treatment, ultrasonic waves act as an energy source for use in intracorporeally accumulating heat energy, and absorption of ultrasonic energy by a living body is closely linked to a treatment effect. In general, using a sound speed c, a medium density ρ, and a sound pressure amplitude p, a magnitude of ultrasonic energy per unit time is expressed as follows:

p2/(ρ·c)

An absolute sound pressure alone is involved irrespective of whether the pressure is positive or negative. If an irradiation time is shorter than the time of intracorporeal thermal diffusion (approximately several min), applied ultrasonic energy is thought to be nearly entirely converted into thermal energy. Using the sound speed c, medium density ρ, sound pressure amplitude p, and irradiation time t, a temperature rise due to ultrasonic irradiation is expressed as follows:

p²/(ρ·c)·t

When the irradiation time is shorter than the time of intracorporeal thermal diffusion, the irradiation time t is made longer. Thus, even when the amplitude is low, a heat coagulation treatment effect can be exerted.

In contrast, as for the former phase change, the phase change is found to be defined mainly with a maximum value of a negative pressure of ultrasonic waves to be irradiated. The negative pressure PNM of ultrasonic waves necessary to induce a phase change is found to have the following relationship to constants k₁ and k₂ and a frequency f:

PNW=k₁−k₂×f

where f>1 MHz and f<10 MHz are satisfied. The relational equation is derived from the results of experiments described below.

An example of the present invention is an ultrasonic wave irradiation device that irradiates ultrasonic waves to a subject who has a contrast medium, which makes a phase change from liquid to gas along with ultrasonic irradiation, administered thereto. The ultrasonic wave irradiation device is characterized by: an ultrasonic wave transmitting/receiving unit that transmits first ultrasonic waves and second ultrasonic waves to the subject, and receives ultrasonic waves from the subject; and the fact that the first ultrasonic waves have a higher frequency than the second ultrasonic waves do, and have a higher or equal maximum negative pressure value, that is, have a maximum negative pressure value equal to or larger than the maximum negative pressure value of the second ultrasonic waves.

### Advantage of the Invention

As described above, according to the present invention, diagnosis or treatment can be achieved in combination with a phase change type ultrasonic wave contrast medium with necessary ultrasonic intensity retained at a necessary minimum level. In particular, interference of diagnostic (phase change) ultrasonic waves with treatment ultrasonic waves can be prevented. Owing to these advantages, a safer diagnosis/treatment technology can be provided.

### BRIEF DESCRIPTION OF THE DRAWINGS

FIG. 1 is a diagram showing an experimental system.

FIG. 2 is a diagram showing a relationship between an ultrasonic maximum negative pressure necessary to induce a phase change of nano-droplets under water, and a ratio of the maximum negative pressure to a maximum positive pressure.

FIG. 3 is a diagram showing a relationship between an ultrasonic maximum negative pressure necessary to induce a phase change of nano-droplets under water, and an ultrasonic frequency.

FIG. 4 is a diagram showing a relationship between an ultrasonic maximum negative pressure necessary to induce a phase change of nano-droplets in a mouse tumor, and a ratio of the maximum negative pressure to a maximum positive pressure.

FIG. 5 is a diagram showing a relationship between an ultrasonic maximum negative pressure necessary to induce a phase change of nano-droplets in a mouse tumor, and an ultrasonic frequency.

FIG. 6 is a diagram showing properties of phase change ultrasonic waves and treatment ultrasonic waves in the present invention.

FIG. 7 is a diagram showing an example of an ultrasonic wave irradiation device of the present invention.

FIG. 8 is a diagram showing an example of the results of irradiation by the ultrasonic wave irradiation device of the present invention.

FIG. 9 is a diagram showing an example of the results of irradiation by the ultrasonic wave irradiation device of the present invention.

### BEST MODE FOR CARRYING OUT THE INVENTION

Studies have demonstrated that an ultrasonic negative pressure PNM necessary to induce a phase change has the following relationship to constants k₁ and k₂ and a frequency f:

PNM=k₁−k₂×f

where f>1 MHz and f<10 MHz are satisfied. This relational equation is derived from the results of experiments described below.

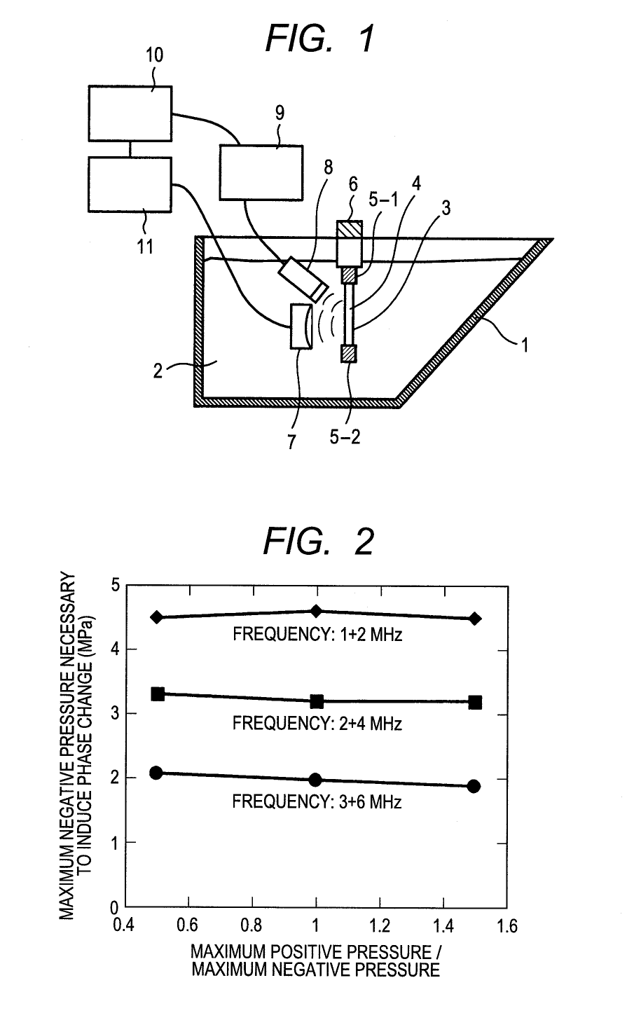

A first example will be described below. This example is to study a change under water in an ultrasonic maximum negative pressure necessary to a phase change when a ratio of a positive pressure to a negative pressure is changed. Using an experimental system shown in FIG. 1, waves having a second harmonic f2 superposed on a fundamental frequency f1 with different phase differences were transmitted to induce a phase change. Magnitudes of maximum negative pressures necessary to induce a phase change when a ratio of a magnitude of a maximum negative pressure of ultrasonic waves to a maximum positive pressure was changed to 0.5, 1, and 2 were investigated under water. In FIG. 1, with a resin water tank 1 filled with deaerated water 2 set to 37° C., a sample 4 enclosed in a sample enclosure tube 3 is locked together with the tube under water using tube end immobilization clips 5 and a sample fixture. A sample-phase change convergent ultrasonic-wave generation transducer 7 is 40 mm in diameter, has an f-number of 1, and is designed to be able to concurrently irradiate ultrasonic waves of any of pairs of frequencies of 1 MHz and 2 MHz, 2 MHz and 4 MHz, and 3 MHz and 6 MHz. The sample 4 is retained at the focus of the transducer 7 by a probe 8 of a phase change observation ultrasonic diagnosis apparatus. While an image is being acquired by an ultrasonic diagnosis apparatus 9, a phase change ultrasonic signal generator 10 and an amplifier 11 are used to irradiate phase-change ultrasonic waves from the ultrasonic transducer 7 for five sec. When the intensity of echoes from the sample is changed to be twice or more larger than it is before irradiation, a decision is made that a phase change has occurred. The pressure of ultrasonic waves is measured by an underwater microphone of 0.5 mm in diameter.

An employed preparation method for nano-droplets will be described below. Components listed below were poured all together. While distilled water of 20 ml was slowly added to the components, it was homogenized in an Ultra-Turrax T25 (Janke & Knukel in Staufen, Germany) at 9500 rpm at ice temperature for one min.

| | |
|---|---|
| glycerol | 2.0 g |
| α-tocopherol | 0.02 g |
| cholesterol | 0.1 g |
| lecithin | 1.0 g |
| perfluoropentane | 0.1 g |
| perfluoroheptane | 0.1 g |

An emulsion obtained through homogenization was subjected to high-pressure emulsification processing in an EmulsiFlex C5 (Avestin in Ottawa, Canada) at 20 MPa for two min, and then filtered by a membrane filter of 0.4 micrometer thick. Through the processing, a nearly transparent micro-emulsion was obtained. Using an LB-550 (Horiba Ltd. in Tokyo), it was confirmed that 98% or more of the obtained micro-emulsion had a diameter of 200 nm or less.

FIG. 2 shows an example of the results. The magnitude of a maximum negative pressure necessary to a phase change varies depending on frequencies. In the case of the highest frequency pair of 3 MHz and 6 MHz, a phase change is induced with the lowest maximum negative pressure. As seen from the drawing, when the frequencies employed are lower, the maximum negative pressure of a higher value is necessary. Even when the ratio of the maximum positive pressure to the maximum negative pressure is changed, the maximum negative pressure necessary to a phase change hardly varies.

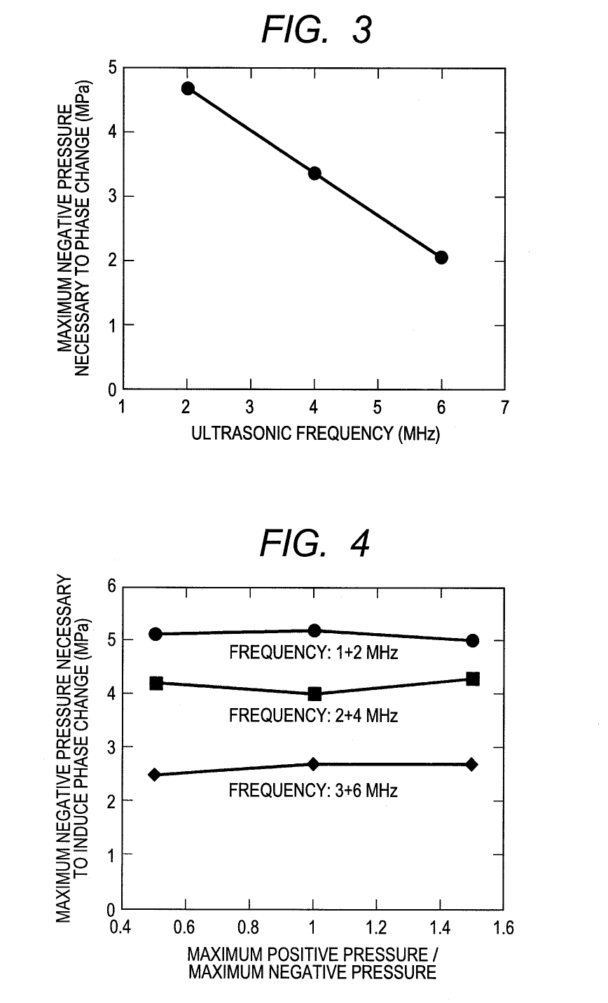

A second example will be described below. This example is to study the dependency under water of an ultrasonic maximum negative pressure, which is necessary to a phase change, on an ultrasonic frequency. FIG. 3 shows the results of investigation performed on an effect, which the ultrasonic frequency to be used for a phase change exerts on a threshold for the phase change, using the experimental system shown in FIG. 1. In this study, as the frequency, sole frequencies of 2 MHz, 4 MHz, and 6 MHz are adopted. The drawing demonstrates that when the ultrasonic frequency gets higher, the maximum negative pressure necessary to the phase change decreases, and that when the frequency gets higher, the maximum negative pressure decreases.

A third example will be described below. This example is to study a change in an ultrasonic maximum negative pressure, which is necessary to a phase change, occurring when a ratio of a positive pressure to a negative pressure is changed. The sample holder 4 was replaced with a mouse retainer in order to adopt an anesthetized mouse as the sample 3 shown in FIG. 1, and verification was performed using the animal. It had been approximately two weeks since the mouse employed in this verification had an experimental tumor Colon26 subcutaneously implanted. The diameter of the tumor was approximately 15 mm. The focus of the phase change ultrasonic transducer 7 was set to a depth of 5 mm from the surface of the mouse tumor, and an ultrasonic maximum negative pressure necessary to a phase change was measured in the same manner as it was in the test example 1. FIG. 4 shows an example of the results. Even in this study, sole frequencies of 2 MHz, 4 MHz, and 6 MHz are employed. Similarly to the test example 1, the magnitude of the maximum negative pressure necessary to a phase change varies depending on the frequency. In the case of the highest frequency pair of 3 MHz and 6 MHz, a phase change is induced with the lowest maximum negative pressure. When the frequency employed is lower, the maximum negative pressure of a higher value is necessary. Incidentally, even when the ratio of the maximum positive pressure to the maximum negative pressure is changed, the maximum negative pressure necessary to a phase change hardly varies.

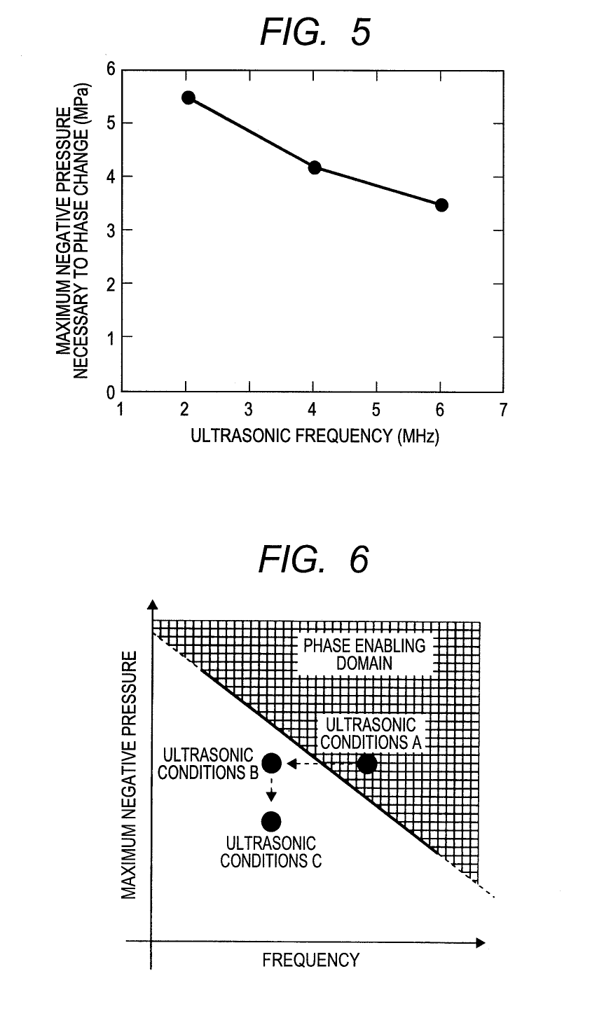

A fourth example will be described below. This example is to study the dependency in a mouse tumor of an ultrasonic maximum negative pressure, which is necessary to a phase change, on an ultrasonic frequency. The sample holder 4 was replaced with a mouse retainer in order to adopt an anesthetized mouse as the sample 3 in FIG. 1, and verification was performed using the animal. FIG. 5 shows the results of investigation performed on an effect which the ultrasonic frequency to be used for a phase change exerts on a threshold for the phase change. In this study, as the frequency, sole frequencies of 2 MHz, 4 MHz, and 6 MHz are adopted. The drawing demonstrates that when the ultrasonic frequency gets higher, the maximum negative pressure necessary to a phase change decreases and that when the frequency gets higher, the maximum negative pressure decreases.

An ultrasonic irradiation time necessary to the foregoing results of studies and phase change generally ranges from several microseconds to several milliseconds. When observed on a scale of a time ranging from several seconds to several tens of seconds and necessary to heat coagulation, ultrasonic waves are almost pulses. From these viewpoints, when a phase change is induced, ultrasonic pulses having the highest possible frequency and maximum negative pressure value are employed. It is found that if ultrasonic pulses having a lower frequency than that employed in order to induce the phase change, and having a smaller maximum negative pressure value than that employed in order to induce the phase change are employed in heat coagulation treatment succeeding occurring of a phase change, an ultrasonic irradiation sequence causing a little interaction between application to a phase change and application to heat coagulation treatment can be realized. Referring to FIG. 6, a description will be made in details. FIG. 6 shows frequencies on the axis of abscissas and maximum negative pressure values on the axis of ordinates. As seen from FIG. 3 and FIG. 5, a domain which is defined with the frequencies and maximum negative pressure values and within which a phase change can be induced (phase change enabling domain) is present. Ultrasonic conditions A are indicated with a point present in the phase change enabling domain. Since ultrasonic conditions B share the same maximum negative pressure value with the ultrasonic conditions A, and include a lower ultrasonic frequency, the ultrasonic conditions B fall outside the phase change enabling domain. Since ultrasonic conditions C share the same frequency with the ultrasonic conditions B and include a smaller maximum negative pressure value, the ultrasonic conditions C fall farther outside the phase change enabling domain than the ultrasonic conditions B do. The ultrasonic conditions A are adopted for application to a phase change, and the ultrasonic conditions B or C are adopted for application to heat coagulation treatment. Thus, the conditions can be regarded as the conditions under which a phase change is induced during heat coagulation treatment.

The present invention is provided based on the foregoing results of studies.

The ultrasonic wave irradiation device in accordance with the present invention includes: means for irradiating phase-change ultrasonic waves to an object area; an image processing unit that produces an ultrasonic diagnostic image showing a situation of production of microbubbles due to a phase change; and means for controlling conditions for irradiation of ultrasonic waves with which heat coagulation treatment is performed based on verification of production of microbubbles using the ultrasonic diagnostic image. Controlling the conditions for irradiation of ultrasonic waves makes it possible to produce ultrasonic waves, which have substantially the same maximum negative pressure value as the maximum negative pressure value of ultrasonic waves employed for a phase change and has a frequency lower than that employed for the phase change, on the basis of verification of production of microbubbles.

When a phase change is not recognized in an object area despite irradiation of phase-change ultrasonic waves, the phase-change ultrasonic waves may be further irradiated by raising the ultrasonic intensity. As the frequency of the ultrasonic waves for a phase change, since it has been demonstrated as shown in FIG. 2 that when the frequency is higher, the phase change is induced with a lower ultrasonic negative pressure, approximately 1 MHz or more is preferred. The intravital absorption rate of ultrasonic waves is nearly proportional to the ultrasonic frequency. At a frequency of 10 MHz, ultrasonic waves reach only a body surface because of attenuation. Therefore, a frequency of roughly about 10 MHz or less is practical. Eventually, as the ultrasonic frequency for a phase change in the present invention, a frequency roughly equal to or larger than about 1 MHz and equal to or smaller than about 10 MHz is preferred.

Embodiments of the present invention will be concretely described below. The present invention is not limited to the embodiments.

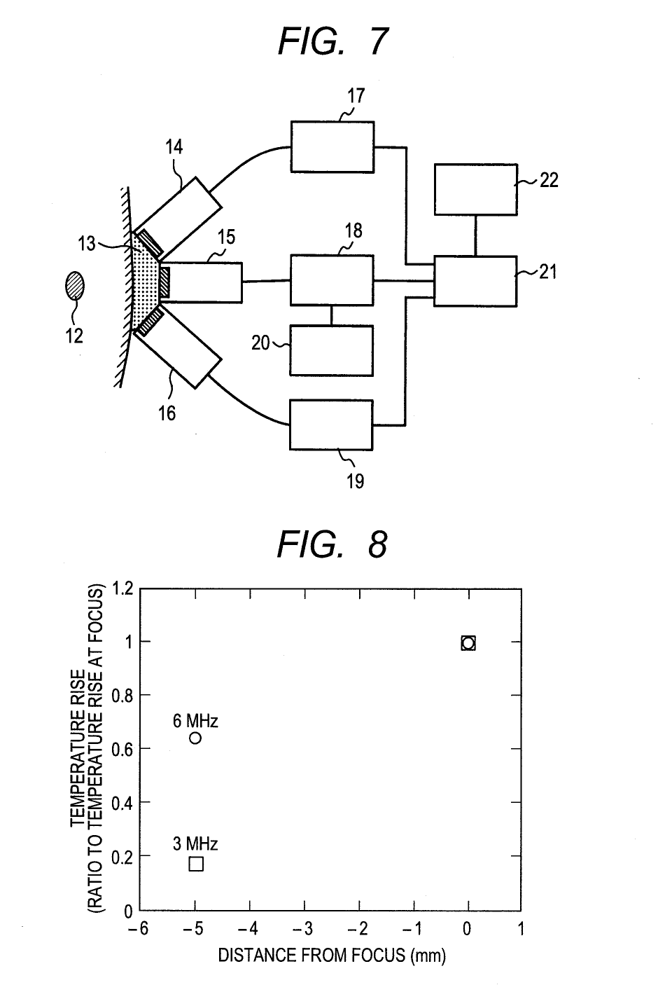

Referring to FIG. 7, an embodiment of the present invention will be described below.

An ultrasonic wave irradiation device of the present embodiment includes a phase-change ultrasonic wave transmission unit 14, a phase-change detection ultrasonic wave transmitting/receiving unit 15, and a treatment ultrasonic wave transmission unit 16 each of which is disposed on an object of treatment 12 via an acoustic coupling material 13, a phase-change ultrasonic wave control unit 17, a treatment ultrasonic wave control unit 18, a phase-change quantification signal processing unit 19, a treatment ultrasonic condition arithmetic unit 20, an image processing unit 21, and an input/display unit 22.

The phase-change ultrasonic wave transmission unit 14 is constructed to be able to irradiate ultrasonic waves that has a sole frequency selected from among the range from 1 MHz to 10 MHz or a frequency selected as a fundamental frequency from among the range from 1 MHz to 5 MHz and a frequency which is a multiple of the fundamental frequency, and that has a maximum negative pressure larger than roughly 0.1 MPa and lower than 10 MPa. As the frequency of ultrasonic waves for a phase change, since it is demonstrated as shown in FIG. 3 and FIG. 5 that when the frequency is higher, a phase change is induced at a lower ultrasonic negative pressure, roughly 1 MHz or more is preferred. In addition, the intravital absorption rate of ultrasonic waves is nearly proportional to the ultrasonic frequency. At the frequency of 10 MHz, ultrasonic waves reach a body surface at most because of attenuation. A frequency of roughly about 10 MHz or less is practical. Therefore, as the ultrasonic frequency for a phase change in the present invention, a frequency roughly equal to or larger than about 1 MHz and equal to or smaller than about 10 MHz is preferred. When a phase change is induced by ultrasonic waves for normal diagnosis, a phase change is induced not only in an aimed region but also an entire range to be observed, and region selective diagnosis and treatment is hindered. Therefore, ultrasonic waves for a phase change preferably exhibit a higher sound pressure than those from an ordinary diagnosis apparatus. Therefore, a negative pressure of roughly 0.1 MPa or more is preferably employed. When consideration is taken into the safety of a living body, the negative pressure should preferably not exceed 10 MPa. The phase-change detection ultrasonic wave transmitting/receiving unit 15 is constructed to be able to transmit or receive ultrasonic waves that have a frequency which roughly ranges from about 2 MHz to about 10 MHz and is employed in an ordinary ultrasonic diagnosis apparatus, and that have an acoustic intensity of a temporal mean intensity of 0.72 W/cm² or less. The treatment ultrasonic wave transmission unit 16 is constructed to be able to irradiate ultrasonic waves of a sole frequency, which is selected from among the range from 0.5 MHz to 10 MHz and used to perform treatment using a heating action of ultrasonic waves, or of a frequency selected as a fundamental frequency from among the range from 0.5 MHz to 5 MHz and a frequency that is a multiple of the fundamental frequency. The acoustic intensity takes on an arbitrary value selected from among the range from 100 W/cm² to 5000 W/cm².

A phase change of a phase change type ultrasonic wave contrast medium in the region to be treated 12 which is derived from ultrasonic irradiation from the phase-change ultrasonic wave transmission unit 14 is detected based on a receiving signal of the phase-change detection ultrasonic wave transmitting/receiving unit 15. The fact that the contrast medium exists in the region to be treated is verified through image processing by the phase-change quantification signal processing unit 19. The ultrasonic irradiation from the treatment ultrasonic wave transmission unit 16 is controlled to obtain the phase-change quantification signal. The phase-change quantification signal processing unit 19 processes a signal for image processing necessary to quantify a change in the intensity or frequency component of an ultrasonic echo signal along with the phase change of the contrast medium. For quantification, a pre-phase change signal recording unit for holding an ultrasonic echo signal obtained before irradiation of phase-change ultrasonic waves, and a post-phase change signal recording unit for holding an ultrasonic echo signal obtained during irradiation of the phase-change ultrasonic waves or after the irradiation are employed, and an arithmetic unit obtains a difference between specific frequency components of the signals held in the respective recording units. In particular, even-numbered harmonics of the center frequency of phase-change detection ultrasonic waves obtained before irradiation of phase-change ultrasonic waves and during the irradiation or after the irradiation are preferably compared with each other. The treatment ultrasonic condition arithmetic unit 20 performs computation for determining conditions for ultrasonic irradiation for treatment on the basis of conditions for ultrasonic irradiation under which formation of a phase change at an intended region quantified by the phase-change quantification signal processing unit 19 exceeds a predefined value.

According to the ultrasonic wave irradiation device of the present embodiment, a phase change or treatment can be induced or performed without causing conditions for ultrasonic irradiation inducing a phase change, and conditions for ultrasonic irradiation, which are employed in treatment, to interfere with each other. For example, a procedure described below is available.

To begin with, a phase-change detection ultrasonic wave transmitting/receiving unit 4 acquires an ultrasonic tomographic image near a region to be treated 1. After a phase change contrast medium is administered, while the phase-change detection ultrasonic wave transmitting/receiving unit 4 acquires the ultrasonic tomographic image, a phase-change ultrasonic wave irradiation unit 3 irradiates phase-change ultrasonic waves (10 waves×50) of 5 MHz and a maximum negative pressure of 3 MPa synchronously with the phase-change detection ultrasonic wave transmitting/receiving unit 4 while scanning substantially the same plane as the plane of the ultrasonic tomographic image acquired by the phase-change detection ultrasonic wave transmitting/receiving unit 4 or part of the plane. When the fact that the echo intensity acquired by the phase-change detection ultrasonic wave transmitting/receiving unit 14 exhibits a rise of a predetermined degree (for example, twice or more, or equal to or larger than a threshold) owing to a phase change of the contrast medium is recognized by the image processing unit 9, the frequency of irradiated ultrasonic waves, maximum negative pressure, number of waves, total irradiation time, and others are posted as ultrasonic conditions for a phase change to the treatment ultrasonic condition arithmetic unit 20. When the rise in the echo intensity obtained by the phase-change detection ultrasonic wave transmitting/receiving unit 4 does not exceed the predetermined value, while the maximum negative pressure is increased in units of a predetermined percentage (for example, in units of 10%), irradiation of phase-change ultrasonic waves is repeated. Although the predetermined upper limit of the maximum negative pressure (for example, 5 MPa) is attained, if a phase change is not induced, the fact is posted to an operator via the input/display unit 22. If a phase change is induced at a maximum negative pressure equal to or smaller than the predetermined upper limit, the treatment ultrasonic condition arithmetic unit 20 performs computation to calculate conditions for ultrasonic irradiation for heat coagulation treatment, which are suitable for treatment, from the conditions for irradiation of phase-change ultrasonic waves. The computation is to perform irradiation at the highest possible acoustic intensity without making the maximum negative pressure larger than that of phase-change ultrasonic waves. Namely, while the maximum negative pressure of treatment ultrasonic waves is made smaller than the negative pressure of phase-change ultrasonic waves, the acoustic intensity is adjusted. For example, the computation brings the frequency to a ½ or less of the frequency of phase-change ultrasonic waves, and makes the maximum negative pressure substantially identical to that of the phase-change ultrasonic waves. In the computation, it is utilized that when the frequency becomes a ½, a threshold necessary to a phase change is, as shown in FIG. 5, raised by approximately 1 MPa or more. The maximum negative pressure value is not decreased in order to shorten a treatment time. As another example, waves having ultrasonic waves, which have a frequency that is a ½ or less of that of phase-change ultrasonic waves and have a maximum negative pressure substantially identical to that of the phase-change ultrasonic waves, and ultrasonic waves, which have a frequency that is a ¼ or less of that of the phase-change ultrasonic waves and has a maximum negative pressure substantially identical to that of the phase-change ultrasonic waves, added up so that the phase difference in an intended region will range from 5/4π to 7/4π may be employed (the maximum negative pressure is nearly equal to that of the phase-change ultrasonic waves and the maximum positive pressure is larger than that of the phase-change ultrasonic waves). In this computation, it is utilized that a phase change is induced dependently on the maximum negative pressure value, while the phase change is induced depending on an absolute maximum sound pressure value during heat coagulation treatment. Although the maximum negative pressure value decreases, the maximum positive pressure value is high. Therefore, a phase change is hardly induced during treatment, and the treatment time is shortened. In addition to or instead of frequency components of a ¼ of the frequency of the phase-change ultrasonic waves, frequency components of ½n (where n denotes a natural number equal to or larger than 3) may be employed. In the computation, it is utilized that a phase change is induced dependently on the maximum negative pressure value, while the phase change is induced dependently on the absolute maximum sound pressure value during heat coagulation treatment. In addition, since the frequency employed is low, treatment ultrasonic waves can reach a deep region. Although the maximum negative pressure value decreases, the maximum positive pressure value is high. Therefore, a phase change is hardly induced during treatment, and the treatment time is shortened. In addition, treatment of the deep region is easy to do.

Next, the region selectivity of a temperature rise in a case where the frequency of treatment ultrasonic waves is a ½ of that of phase-change ones and a maximum negative pressure thereof is substantially identical to that of the phase-change ones will be discussed below.

The experimental system shown in FIG. 1 was used to administer nano-droplets by 0.1 ml to a mouse which had an experimental tumor Colon26 subcutaneously implanted therein and grown to have a diameter of 20 mm. In fifteen min, a phase change was induced as phase-change ultrasonic waves at a frequency of 6 MHz and a maximum negative pressure of 3 MPa (four waves, 48 times/sec, 5 sec). Further, temperature rises at the focus and at a point 5 mm in front of the focus occurring when heating was performed for 20 sec at a frequency of 3 MHz and a maximum negative pressure of 3 MPa were measured by a K-type thermocouple of 0.1 mm in diameter, and maximum temperature rises were obtained. FIG. 8 shows an example of the results. As collative results, results obtained by performing treatment at a frequency of 6 MHz and a maximum negative pressure of 3 MPa are shown together. In the case of using 6 MHz, the temperature rise at the region 5 mm in front of the focus is approximately 65% of that at the focus. In the case of using 3 MHz, the temperature rise is approximately 20%. Compared with the case of 6 MHz, the temperature rise apparently selectively occurs at the focus.

Next, the region selectivity of a temperature rise in a case where the frequency of treatment ultrasonic waves is a ½ of that for phase-change ones, the maximum negative pressure thereof is substantially identical to that of the phase-change ones, and the frequency of 1 of the phase-change ones is superposed with a phase difference varied will be discussed below.

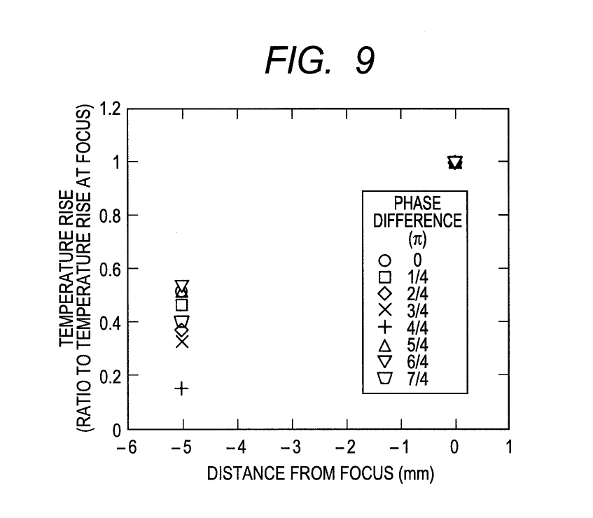

The experimental system shown in FIG. 1 was used to administer nano-droplets by 0.1 ml to a mouse which had an experimental tumor Colon26 subcutaneously implemented therein and grown to have a diameter of 20 mm. In fifteen min, a phase change was induced as phase-change ultrasonic waves at a frequency of 8 MHz and a maximum negative pressure of 3 MPa (four waves, 48 times per sec, 5 sec). Further, temperature rises at the focus and a point 5 mm in front of the focus occurring when heating was performed for 20 sec with synthetic waves of waves having a frequency of 4 MHz and a maximum negative pressure of 3 MPa and waves having a frequency of 2 MHz and a maximum negative pressure of 3 MPa were measured by a K-type thermocouple of 0.1 mm in diameter, and maximum temperature rises were obtained. FIG. 9 shows an example of the results in a case where the relative phase difference between 4 MHz and 2 MHz of the synthetic waves is varied. Only when the relative phase difference is π (4/4π in the drawing), the temperature rise at the region 5 mm in front of the focus is as low as approximately 20% of that at the focus. With any other phase difference, the ratio ranges from approximately 40% to approximately 60%. Apparently, a difference from the temperature rise at the focus is not outstanding.

### DESCRIPTION OF REFERENCE NUMERALS

1 resin water tank
2 deaerated water set at 37° C.
3 sample enclosure tube
4 sample
5 tube end immobilization clip
6 sample fixture
7 transducer for generation of sample phase-change convergent ultrasonic waves
8 probe of a phase-change observation ultrasonic diagnosis system
9 ultrasonic diagnosis system
10 phase-change ultrasonic signal generator
11 amplifier
12 object of treatment
13 acoustic coupling agent
14 phase-change ultrasonic wave transmission unit
15 phase-change detection ultrasonic wave transmitting/receiving unit
16 treatment ultrasonic wave transmission unit
17 phase-change ultrasonic wave control unit
18 treatment ultrasonic wave control unit
19 phase-change quantification signal processing unit
20 treatment ultrasonic condition arithmetic unit
21 image processing unit
22 input/rendering unit.

1. An ultrasonic wave irradiation device that irradiates ultrasonic waves to a subject who has a contrast medium, which makes a phase change from liquid to gas along with ultrasonic irradiation, administered thereto, characterized in that:
the ultrasonic wave irradiation device comprises an ultrasonic wave transmitting/receiving unit that transmits first ultrasonic waves and second ultrasonic waves to the subject and receives ultrasonic waves from the subject; and
the frequency of the first ultrasonic waves is higher than that of the second ultrasonic waves, and the maximum negative pressure value thereof is equal to or larger than the maximum negative pressure value of the second ultrasonic waves.

2. The ultrasonic wave irradiation device according to claim 1, characterized in that the first ultrasonic waves are contrast-medium phase-change ultrasonic waves, and the second ultrasonic waves are treatment ultrasonic waves.

3. The ultrasonic wave irradiation device according to claim 1, characterized in that the ultrasonic wave irradiation device further comprises a second ultrasonic condition arithmetic unit that computes conditions for irradiation of the second ultrasonic waves on the basis of a signal received by the ultrasonic wave transmitting/receiving unit.

4. The ultrasonic wave irradiation device according to claim 1, characterized in that the ultrasonic wave transmitting/receiving unit causes the second ultrasonic waves to have a maximum negative pressure which is smaller than the negative pressure of the first ultrasonic waves.

5. The ultrasonic wave irradiation device according to claim 1, characterized in that the ultrasonic wave transmitting/receiving unit adjusts the acoustic intensity while causing the second ultrasonic waves to have a maximum negative pressure which is smaller than the negative pressure of the first ultrasonic waves.

6. The ultrasonic wave irradiation device according to claim 1, characterized in that the ultrasonic wave irradiation device further comprises an image processing unit which produces an image to be used to verify the phase change on the basis of a receiving signal of the ultrasonic wave transmitting/receiving unit.

7. The ultrasonic wave irradiation device according to claim 6, characterized in that the ultrasonic wave irradiation device further comprises a second ultrasonic condition arithmetic unit that computes conditions for irradiation of the second ultrasonic waves on the basis of the verification of the image.

8. The ultrasonic wave irradiation device according to claim 1, characterized in that the ultrasonic wave transmitting/receiving unit causes the second ultrasonic waves to have a frequency that is a ½ or less of the frequency of the first ultrasonic waves.

9. The ultrasonic wave irradiation device according to claim 1, characterized in that the ultrasonic wave transmitting/receiving unit causes the second ultrasonic waves to have a frequency which is a ½ or less of the frequency of the first ultrasonic waves, and a maximum negative pressure which is substantially identical to that of the first ultrasonic waves.

10. The ultrasonic wave irradiation device according to claim 1, characterized in that the ultrasonic wave transmitting/receiving unit makes the frequency of the first ultrasonic waves equal to or larger than 1 MHz and equal to or smaller than 10 MHz.

11. The ultrasonic wave irradiation device according to claim 1, characterized in that the ultrasonic wave transmitting/receiving unit brings the second ultrasonic waves to synthetic waves of sound waves, the frequency of which is a ½ or less of the frequency of the first ultrasonic waves, and sound waves whose frequency is a ½n (where n denotes an integer equal to or larger than 1) of the frequency of the first ultrasonic waves.
